(19) United States
(12) Patent Application Publication
(10) Pub. No.: US 2006/0029576 A1
(43) Pub. Date: Feb. 9, 2006

(54) Biological control of pythium disease in crops

(75) Inventors: Hung Chang Huang, Lethbridge (CA); Sylvie D. Bardin, Oshawa (CA); Russell Scott Erickson, Raymond (CA)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(73) Assignee: HER MAJESTY THE QUEEN IN RIGHT OF CANADA, THE MINISTER OF AGRICULTURE AND AGRI-FOOD, Ottawa (CA)

(21) Appl. No.: 10/910,834

(22) Filed: Aug. 4, 2004

(51) Int. Cl. A01N 63/00; C12N 1/20

(52) U.S. Cl. 424/93.4; 435/252.2

(57) ABSTRACT

Strains of Rhizobium leguminosarum biovar viceae have antifungal activity against the pathogen Pythium ultimum. Compositions and methods for treating or protecting plants susceptible to Pythium ultimum damage, and Pythium sp. “group G” damage in particular, are provided. Such strains include, for example, the strains deposited in the International Depository Authority of Canada under accession numbers IDAC 200704-01, IDAC 200704-02, IDAC 200704-03, and IDAC 200704-04.

### FIELD OF THE INVENTION

The invention relates to control of crop disease caused by the fungus Pythium, and compositions and methods therefore. In particular, the invention relates to the preparation and application of biocontrol agents from novel strains of Rhizobium leguminosarum biovar viceae affective in controlling Pythium disease.

### BACKGROUND OF THE INVENTION

“Damping-off” is the sudden plant death in the seedling stage due to the attack of fungal pathogens such as Pythium spp. and Rhizoctonia solani. The pathogens are soilborne and are stimulated to grow and infect the seed or seedling of host crops by nutrients released from a germinating seed. Damping-off disease of seedlings occurs in most soils, temperate and tropical climates, and in greenhouses. The disease affects seeds and seedlings of various crops grown under greenhouse and/or field conditions. The amount of damage the disease causes to seedlings depends on the fungus, host tolerance/susceptibility soil moisture, and temperature. Normally, however, cool wet soils favor development of the disease caused by Pythium spp. Roots may rot, or the hypocotyls (lower stem) may either collapse or become wiry. Seedlings may die before or after they emerge from the soil (pre-emergence and post-emergence damping-off, respectively). Seedlings in seedbeds often are completely destroyed by damping-off, or they die after transplanting. Severe loses of plants due to pre- and post-emergence damping-off often results in poor stands of many crops.

Pythium spp. are the causal agents of seed, root, and crown rot diseases of economically important crops worldwide. Pythium sp. “group G”, a sterile form of Pythium ultimum Trow, is a major plant pathogen of numerous crops grown in southern Alberta, including sugar beet (Beta vulgaris L.) and field pea (Pisum sativum L.). Indoor experiments, using soil artificially inoculated with Pythium sp. “group G”, showed that safflower (Carthamus tinctorius L.), canola (Brassica rapa L.), field pea and sugar beet are highly susceptible to the pathogen (Huang et al 1992). Field surveys showed that Pythium spp. were the main cause of poor stands of sugar beet in southern Alberta (Bardin and Huang 2001). Pythium ultimum Trow and Pythium irregulare Buisman were the principal pathogens causing seed rot and damping-off of field pea and reduced seedling establishment in the northern Canadian prairies (Hwang and Chang 1989). Pythium diseases in field crops are usually controlled by seed treatment with fungicides such as Thiram™ 75 WP and (or) Apron™.

Increased health and environmental concerns with the use of chemical fungicides have stimulated the search for alternative ways to control the disease using antagonistic microorganisms as biological control agents. Considerable research has been conducted on biological control of Pythium species using antagonistic bacteria and fungi (Martin and Loper 1999). Satisfactory biocontrol of Pythium damping-off has been achieved using seed treatment with rhizobacteria that are antagonistic to the pathogen (Bardin et al. 2003).

Rhizobium spp. are soilborne bacteria that can establish a symbiotic relationship with legume plants. The symbiosis takes place in plant root nodules, in which the differentiated rhizobia known as bacteroids convert atmospheric nitrogen to a nitrogenous compound that can be used by the plant. Rhizobium species are host specific. For instance, Rhizobium leguminosarum bv. viceae Frank nodulates only plants from the genera Pisum, Lens, Vicia, and Lathyrus. Inoculation of legume seeds with Rhizobium prior to planting is commonly used to improve legume crop production by increasing nodulation, thereby reducing the need for application of nitrogen fertilizer (Brockwell et al. 1995).

Several reports have indicated that Rhizobium and Bradyrhizobium have potential as biocontrol agents of plant pathogens. Rhizobia inhibited mycelial growth of plant pathogens such as Aphanomyces euteiches, Phoma medicaginis (Dileep Kumar et al. 2001), Macrophomina phaseolina, Rhizoctonia solani (Omar and Abd-Alla 1998), Phytophthora cactorum (Drapeau et al. 1973), Fusarium spp. (Drapeau et al. 1973; Omar and Abd-Alla 1998; Dileep Kumar et al. 2001), and P. ultimum (Ozkoc and Deliveli 2001). In addition to in vitro inhibition, some Rhizobium strains reduced disease severity caused by Phytophthora clandestina (Simpfendorfer et al. 1999), as well as Fusarium solani, M. phaseolina, and Rhizoctonia solani (Siddiqui et al. 2000), in greenhouse experiments in which soil was artificially infested with the pathogen. In other studies, Rhizobium inoculation effectively suppressed diseases caused by F. solani (Estevez de Jensen et al. 2002), Fusarium oxysporum, Rhizoctonia bataticola, and Pythium sp. (Nautiyal 1997) in soil naturally infested with these pathogens.

What is needed are biocontrol agents for Pythium spp., particularly biocontrol agents that will protect the crops from disease caused by Pythium ultimum.

### SUMMARY OF THE INVENTION

According to the present invention, strains of the nitrogen-fixing bacteria Rhizobium leguminosarum biovar viceae are utilized to control Pythium infection on crops. The invention relates in particular to microbial pure cultures of four such strains, identified herein as R5, R12, R20 and R21, which were deposited on Jul. 20, 2004 with the International Depository Authority of Canada (IDAC), 1015 Arlington Street, Winnipeg, Manitoba, R3E 3R2, Canada, under the auspices of the Budapest Treaty, under the following IDAC Deposit Accession numbers:

| | |
|---|---|
| R5: | IDAC 200704-04; |
| R12: | IDAC 200704-03; |
| R20: | IDAC 200704-02; |
| R21: | IDAC 200704-01. |

According to one embodiment, the invention provides an antifungal composition comprising bacteria of at least one isolated Rhizobium leguminosarum biovar viceae strain effective in inhibiting growth of Pythium ultimum.

According to another embodiment, the invention is directed to a method for treating or protecting a susceptible plant from Pythium ultimum. The plant or part thereof, or soil surrounding the plant, is contacted with an effective amount of at least one Rhizobium leguminosarum biovar viceae strain which has suppressive activity against Pythium ultimum.

The bacterial strains may be selected on the basis of their ability to inhibit the colonization of Pythium ultimum on an agar plate. When a bacterial strain is said to “inhibit the colonization of Pythium ultimum on an agar plate” means that no mycelial growth of the fungus occurs on a streak of the bacterial strain laid down four centimeters distant from a Pythium ultimum-colonized potato dextrose agar plug on the plate, following incubation of the plate at room temperature for five days. The assay technique is described in more detail below.

According to preferred embodiments of the invention, the Rhizobium leguminosarum biovar viceae strain is effective in inhibiting growth of Pythium sp. “group G”, a sterile form of Pythium ultimum, and the strains are selected on the basis of their ability to inhibit the colonization of Pythium sp. “group G” on an agar plate. Plants susceptible to Pythium sp. “group G” are treated or protected.

As used in the specification and claims, the singular form “a,” “an” and “the” include plural references unless the context clearly dictates otherwise. For example, the term “a cell” includes a plurality of cells, including mixtures thereof.

“Antifungal” means the ability to inhibit the growth of or kill fungi. It should be noted that a biological control agent can act in an antifungal manner by not only exerting a direct effect on a fungal pathogen, but also in an indirect manner, such as by competing with the pathogen for nutrient. Both such direct and indirect actions are understood to be “antifungal”.

As used herein, “biovar” or “biological variant” (or the abbreviation “bv.”) means a strain of a bacterium that is differentiated by biochemical or other non-serological means from another strain. A “strain” is a subset of bacterial species differing from other bacteria of the same species by some minor but identifiable difference.

As used herein, “biological control” is defined as control of a pathogen or insect by the use of a second organism.

As used herein, the term “comprising” is intended to mean that the compositions and methods include the recited elements, but not excluding others.

The term “culturing” refers to the propagation of organisms on or in media of various kinds.

A “composition” is intended to mean a combination of active agent and another compound or composition, inert (for example, a detectable agent or label) or active, such as an adjuvant.

An “effective amount” is an amount sufficient to effect beneficial or desired results. An effective amount can be applied in one or more applications. In terms of treatment and protection, an “effective amount” is an amount sufficient to ameliorate, stabilize, reverse, slow or delay progression of a fungal infection.

An “isolate” is a pure culture derived from a heterogeneous, wild population of microorganisms.

The term “isolated” is used interchangeably with “biologically pure” and means separated from constituents, cellular and otherwise, in which the strain or metabolite is normally associated with in nature.

As used herein, “Pythium ultimum” is meant to include all forms of the species of this name, including but not limited to Pythium sp. “group G”.

By “suppressive activity” of a biological control agent against a fungal pathogen is meant the ability of the agent to ameliorate, stabilize, reverse, slow or delay progression of an infection by the fungal pathogen.

“Whole broth culture” refers to a liquid culture containing both cells and media.

### DETAILED DESCRIPTION OF THE INVENTION

We have found that Pythium diseases may be controlled by strains of the nitrogen-fixing bacteria Rhizobium leguminosarum bv. viceae. In one study, fifty-six percent of strains of R. leguminosarum bv. viceae obtained from field pea and lentil nodules were found to improve emergence of sugar beet seedlings in soil artificially infested with the pathogen Pythium sp. “group G” in indoor experiments. Strains tested as seed treatments in diseased fields effectively controlled damping-off of sugar beet and field pea caused by Pythium spp. The effectiveness of the Rhizobium strains is similar to that of Pseudomonas fluorescens Migula LRC 708, a biological control agent against Pythium damping-off of sugar beet, field pea, canola, and safflower (Bardin et al. 2003). The biological control activity of the Rhizobium strains is not host dependent, as they are effective agents for both legume (pea) and nonlegume (sugar beet) plants.

According to the present invention, R. leguminosarum bv. viceae strains are utilized to control or alleviate Pythium infection on crops. Rhizobium leguminosarum bv. viceae strains are useful for promoting plant health by protecting inoculated seeds from attack by Pythium ultimum, and Pythium sp. “group G” in particular, thereby reducing incidence of damping-off diseases.

The bacteria Rhizobium leguminosarum bv. viceae comprising the active agent of the invention may be isolated from root nodules of host legumes such as pea and lentil. The bacteria are collected from root nodules which are removed from the plants and crushed. The nodule contents are plated on an appropriate medium to support the growth of the bacteria. The bacteria are cultured under conditions favoring the growth thereof. Conditions for culturing Rhizobium leguminosarum strains are known to those skilled in the art, and are exemplified in the Examples which follow. Colonies are isolated from the culture.

The antagonistic activity of isolates against Pythium ultimum may be determined using the dual-culture technique. Each bacterial strain is streaked on a medium which will support the growth of Pythium ultimum, e.g., Tryptone Yeast Extract agar near the edge of a Petri dish (9 cm diameter). After incubation for one day at room temperature, a mycelial plug (0.6 mm diameter) of Pythium ultimum from a 48-hour culture grown on potato dextrose agar is placed in the center of each dish containing the bacterial streaks. The plates are incubated for five days at room temperature (20±2° C.) and the inhibitory effect of each bacterial strain is determined by measuring the inhibition zone of mycelial growth. The inhibitory effect is scored as positive where the Pythium growth stops on or before the bacterial streak line.

The bacteria may be utilized in the form of cultures of bacteria, such as a suspension in a whole broth culture, to prepare appropriate compositions for ground treatment, plant treatment, soil and/or growing media treatment, or seed treatment.

The compositions containing the bacteria as the sole active ingredient, or as a combination with one or more other active ingredients, are prepared in known manner, such as by using standard fermentation methods, processes and equipment, followed by homogeneously mixing and/or grinding the active ingredients with extenders, growth media ingredients (for example such as nutrients, stabilizers, buffering systems, plant growth hormones, and pH adjustment ingredients) and liquid or dry organic or inorganic carriers. Suitable carriers include sterilized and sanitized liquid carriers; pre-sterilized (irradiated or steam sterilized) and non-sterilized peat powders; granulated, spheronized or pelletized peat, clay; and other extenders, filler pigments or minerals. Other carriers for granular formation include talc, gypsum, kaolin, attapulgite, montmorillonite, bentonite, wood flour, ground corn cob grits, starch, cellulose, and bran. The formulations can also contain additives such as adhesives, stickers, binders, polymers and other adjuvants applicable to agricultural or horticultural applications. Stickers or binders may comprise, for example, ethylene glycol, mineral oil, polypropylene glycol, polyvinylacetate, lignosulfonate, polyvinyl alcohol, polyvinylpyrrolidone, graphite, gum Arabic, methyl cellulose, and sucrose.

The compositions of this invention can be formulated in powder or granular form by mixing together all the components, including any carrier and/or other additive(s) which may be utilized until a homogeneous mixture is formed. A sticker, if employed, may then be added and the entire mass mixed again until it has become essentially uniform in composition. The composition may or may not be formulated in a pre-sterilized carrier system.

The optimum concentration of Rhizobium leguminosarum bv. viceae employed in the compositions of the invention for a particular application can be readily determined by those skilled in the art. In general, the concentration of bacteria can range from about 0.001 to about 1%, preferably from about 0.01 to about 0.5%, more preferably from about 0.05 to about 0.1%, by weight.

In the case of a liquid formulation, an aqueous liquid nutrient medium may be utilized, optionally comprising adjuvants such as stickers, stabilizers and colorants.

The composition of the invention may contain, as additional active agents, cells, spores or propagules of other biological control agents, one or more chemical fungicides, or one or more other pesticidal materials, such as insecticides. The chemical fungicide may be selected on the basis of its activity against Pythium spp., or may be selected on the basis of activity against other fungal pathogens.

The method of the invention comprises applying to plants an antifungal effective amount of a composition containing Rhizobium leguminosarum bv. viceae. The composition is most advantageously applied to roots or seeds. The composition may also be utilized as ground treatments in fields or greenhouses. They may be applied to soil surrounding plants, or applied to soil into which seeds or seedlings are planted.

The compositions are applied by methods which include, for example, seed treatments, spray applications, in-furrow applications, soil and growing media inoculation, application through irrigation system, and the like. The compositions can be applied as stand alone or with the standard chemical treatments to control Pythium.

When employed as a seed dressing, the amount of composition is applied such that the seed is coated with a concentration of bacteria adequate to provide protection against Pythium spp. The actual amount to utilize depends on the nature and size of the seed, the amount of the protection desired, the local soil conditions, and other factors which may be taken into account in selecting the appropriate dosage of bacteria. Appropriate application rates of bacteria in terms of colony forming units (cfu) per seed are as follows: large seeded crops—from about 10⁴ to about 10⁸ for legumes, and from about 10⁶ to about 10⁷ for non-legumes; medium size-seeded crops—from about 10³ to about 10⁷ for legumes, and from about 10⁵ to about 10⁶ for non-legumes; small seeded crops—from about 10² to about 10⁶ for legumes, and from about 10⁴ to about 10⁵ for non-legumes. Greater concentrations of bacteria may be applied.

Seeds may be bacterized according to the present invention by steeping the seeds in a suspension of bacteria for an appropriate time, e.g. one hour. According to one such technique, bacterial slurries are prepared by adding 3 ml of 1% methyl cellulose to tryptone yeast medium plates on which the culture is grown, and gently scraping the culture off the plate. For seed treatment, seeds are soaked for, e.g., 20 minutes in the bacterial slurry. The bacterial concentration in the slurry may be determined by plating serial dilutions on tryptone yeast medium (Beringer, 1974).

According to one embodiment, an inoculant composition may be prepared for application to seeds, using ground peat. Methods for the preparation of inoculant compositions of Rhizobium sp. for inoculation of crops, e.g., legumes, to increase nitrogen fixation are known. See, e.g., U.S. Pat. No. 5,484,464, the entire disclosure of which is incorporated herein by reference. One such inoculant composition is prepared from sterilized powdered peat with a moisture content of 6-20%, with or without a sticker. Using aseptic techniques, a suspension of Rhizobium leguminosarum bv. viceae is added to the peat at a rate of from about 10⁵ to about 10⁸ colony forming units of bacteria per gram of peat.

The composition may be utilized to protect any crop which is susceptible to infection and damage by Pythium ultimum, and Pythium sp. “group G” in particular. Such crop species include, for example, sugar beet (Beta vulgaris L.), field pea (Pisum sativum L.), lentil (Lens spp.), safflower (Carthamus tinctorius L.), canola (Brassica rapa L. and Brassica napus L.), chickpea (Cicer spp.), sunflower (Helianthus spp.), alfalfa (Medicago spp.), soybean (Glycine spp.), and field bean (Vicia faba).

### EXAMPLES

In the following Examples, the viable counts of bacterial agents in slurries and on seeds were expressed as mean cfu±SE. Shoot dry mass and emergence data of both indoor and field experiments were analyzed statistically using the Statistic Analysis Software package Version 6.0.9 (Examples 1-6) or Version 8.2 (Example 7) (SAS Institute Inc., Cary, N.C.). Analysis of variance was done using the general linear model procedure. Differences between treatments were analyzed using Fisher's least significant difference (LSD) test. All analyses were performed at the P=0.05 level.

### Example 1

### Isolation of Rhizobium Strains

Strains of R. leguminosarum bv. viceae were isolated from root nodules of field pea and lentil grown in southern Alberta, Canada, as follows. Roots from two plants per crop were washed in water to remove soil particles. The nodules were excised, surface sterilized in 2% sodium hypochlorite for 1 min, washed eight times in sterile distilled water, and crushed with a sterile spatula in 200 μL sterile water. The nodule contents were plated on tryptone-yeast extract medium (TY; Beringer 1974) containing 1.5% agar (Difco, Detroit, Mich.). Following incubation for 3-4 days at room temperature (20±2° C.), a colony from each plate was purified by three successive single colony isolations. Eighteen strains of R. leguminosarum bv. viceae were isolated in this manner. Ten strains were isolated from field pea root nodules and eight from lentil root nodules. Of these strains, 8 showed no potential for control of Pythium damping-off of sugar beet in preliminary indoor experiments and were not tested further. The identities of the 10 remaining strains, 8 from pea and 2 from lentil (Table 1), were confirmed by performing plant nodulation experiments (see below) and by streaking the bacteria on Luria-Bertani (L B, Miller 1972) agar. The strains did not grow on LB, which is consistent with the fact that R. leguminosarum is sensitive to the high salt concentration contained in this media.

### Example 2

### Plant Nodulation by Rhizobium Strains

The ability of the ten Pythium-antagonizing Rhizobium isolates to form nitrogen-fixing nodules on pea and lentil plants was determined in a nitrogen-free medium. Seeds were surface sterilized for 5 min in 50% aqueous sodium hypochlorite, washed 8-10 times with sterile distilled water, and germinated for 2 days in the dark on water agar (1.5%) in Petri dishes. Six seeds were planted in each sterile Leonard jar assembly (Leonard 1943), containing a mixture of quartz sand and vermiculite (1:1; v/v) saturated with nitrogen-free Jensen's nutrient solution (Vincent 1970). Two days after planting the seeds, each jar was inoculated with 10 mL of an aqueous bacterial suspension (10⁷-10⁸ cfu/10 mL) of Rhizobium or with 10 mL water for the uninoculated control. Each treatment was performed in duplicate. The experiment was repeated once. The plants were kept in a growth cabinet in a 16 h light (20° C.): 8 h dark (15° C.) cycle. They were watered with sterile distilled water as required. Lentil and pea plants were collected 26 and 27 days after inoculation, respectively. The shoots of the plants were excised, dried in a 60° C. oven for 5 days, and weighed to determine nodulation efficacy.

Plants inoculated with each of the ten Pythium-antagonizing Rhizobium strains were green and healthy compared with the brown and stunted plants of the uninoculated control. There were pinkish nodules formed on the roots of plants inoculated with the strains, while no nodules developed on the roots of uninoculated plants. In addition, the dry shoot masses of the inoculated plants were significantly (P<0.05) greater than those of uninoculated plants (Table 1). Strain R12 was effective in establishing a beneficial symbiotic interaction with both lentil and pea plants (Table 1).

TABLE 1
Source of Rhizobium leguminosarum bv. viceae strains and
their nodulation efficacy on field pea and lentil.

| | |
|---|---|
| Rhizobium | Shoot dry mass |
| leguminosarum | (% control) |

| bv. viceae* | Plant source† | Pea | Lentil |
|---|---|---|---|
| Strain | | | |
| R3 | Pisum sativum | 245a‡ | nd |
| R4 | Pisum sativum | 243a§ | nd |
| R5 | Pisum sativum | 295a‡ | nd |
| R7 | Pisum sativum | 288a‡ | nd |
| R8 | Pisum sativum | 263a‡ | nd |
| R9 | Pisum sativum | 294a‡ | nd |
| R12 | Lens culinaris | 273a‡ | 306a¶ |
| R19 | Lens culinaris | nd | 327a¶ |
| R20 | Pisum sativum | 235a§ | nd |
| R21 | Pisum sativum | 224a§ | nd |
| Uninoculated control | | 100b | 100b |

Note:
Nodulation efficacy is expressed as percent increase in shoot dry mass of a pea or lentil plant inoculated with a R. leguminosarum bv. viceae strain compared with the uninoculated control (100%). The values represent the means of 12 plants (two pots of 6 plants each) from two independent experiments. Means within the same column followed by the same letter are not significantly different at P = 0.05 level (Fisher's LSD test).
nd, not determined.
*Strains of R. leguminosarum bv. viceae isolated from the root nodules of pea or lentil plants collected in southern Alberta.
†Plant nodules where the bacteria were isolated.
‡Dry shoot mass of the uninoculated control was 202.3 mg/plant.
§Dry shoot mass of the uninoculated control was 271.0 mg/plant.
¶Dry shoot mass of the uninoculated control was 83.0 mg/plant.

### Example 3

### Control of Pythium Damping-Off of Sugar Beet by Rhizobium Strains (Dual Culture Experiments)

The antagonistic activity of the ten remaining R. leguminosarum bv. viceae strains against Pythium sp. “group G” strain LRC 2105 (Huang et al. 1992) was determined by streaking a Rhizobium strain 4 cm away from a potato dextrose agar (PDA) plug colonized by Pythium on TY agar plates (dual culture technique). After incubation at room temperature for 5 days, the inhibitory activity of the Rhizobium strain was determined by measuring the zone of mycelial growth inhibition around the bacterial streak. Three ratings were used: −, no inhibition zone and growth of Pythium over the bacterial streak; +, no inhibition zone, but no growth of Pythium on the bacteria streak; and ++, 1-5 mm inhibition zone. There were three replicates for each treatment and the experiment was repeated once. Strain R5 was rated as ++. It was the only strain showing antagonistic effects to Pythium sp. “group G”, with formation of a small zone of inhibition 2 mm in size. The other 9 strains did not exhibit zones of inhibition but were able to prevent colonization of the bacterial streak by the pathogen and were therefore rated as + (slight inhibition).

### Example 4

### Test for Protease Production by Rhizobium Strains

Protease production was determined by incubating colonies of R. leguminosarum bv. viceae on skim milk agar plates (Dunne et al. 1997) for 5 days at room temperature (20±2° C.). Protease activity was compared with the protease positive strain, Pseudomonas fluorescens Migula LRC 708, which degrades casein and causes clearing of the skim milk agar plate (Bardin et al. 2003). There were three replicates for each treatment and the experiment was repeated once. Unlike strain P. fluorescens 708, none of the Rhizobium strains tested showed protease activity, as they failed to produce clearing zones around the colonies when plated on the skim milk agar plates. Thus, production of extracellular proteases is not the mechanism of action of the Rhizobium strains.

### Example 5

### Seed Treatment by Rhizobium Strains (Indoor Experiments)

The strains of R. leguminosarum bv. viceae were further tested as seed treatments for control of Pythium damping-off of sugar beet in nonsterile soil. Bacterial cultures were grown on TY agar in Petri dishes (5.5 cm in diameter) for 48 hours at room temperature. The bacterial culture was resuspended in 3 mL of 1% methyl cellulose (MC) (Aldrich Chemical, Milwaukee, Wis.) by scraping the agar surface gently with a spatula. This resulted in bacterial slurries with a concentration averaging 3.9×10⁹±0.5×10⁹ (mean±SE) cfu/mL. Sugar beet (Beta vulgaris ‘HM Bergen’) (Novartis Seeds—Hilleshög, Longmont, Colo.) seeds were soaked for 20 minutes in the MC-bacterial slurry and were seeded directly into soil artificially infested with Pythium sp. “group G” strain LRC 2105. The soil consisted of 3 parts topsoil (Bzdell Soil Service, Lethbridge, Alberta.), 1 part sand (Tollestrup Construction, Lethbridge, Alberta), and 1 part peat moss (Premier Horticulture, Red Hill, Pa.). The Pythium inoculant was prepared in pans containing a sterile mixture of 150 g wheat bran (Ellison Milling, Lethbridge, Alberta), 150 g corn meal (McCormick, London, Ontario), and 300 mL distilled water. Twenty plugs (8 mm in diameter) of a 48-hour-old PDA culture of Pythium sp. “group G” were placed in each pan. After incubation for 2 weeks at room temperature in the dark, the wheat bran-corn meal mix was completely colonized by the pathogen. The Pythium inoculum was air-dried at room temperature for 4 days and ground using a Thomas-Wiley model 4 laboratory mill (Thomas Scientific, Philadelphia, Pa.) equipped with a 1-mm mesh screen. The soil, artificially infested with Pythium sp. “group G” at a concentration of 2 g inoculum/kg soil, was used to fill root trainers (Spencer-Lemaire Industries, Edmonton, Alta.), each containing 17 books of six cells per book. One sugar beet seed was planted per root trainer cell at a depth of 1.5 cm. Uninoculated seeds were also planted in non-infested soil. The root trainers were soaked in a water-filled tray until the soil was saturated by capillary action, and were then placed in propagator trays (The Stewart Company, Croydon, Surrey, UK) to create a high-moisture environment. The propagator trays were kept in a growth chamber in a 16 hour light (20° C.): 8 hour dark (15° C.) cycle. In each experiment there were three replicates per treatment and 18 seeds per replicate. The treatments were arranged in a completely randomized design. Seedling emergence was recorded 14 days after planting, and data from bacterial seed treatments were compared with the uninoculated control. Each set of experiments was repeated twice. Non-germinated seeds were collected, washed with sterile water, surface sterilized in 70% ethanol for 2 min, and plated on PDA in Petri dishes. The fungi isolated from the seeds were purified on PDA, and the genus of each fungus isolated was determined based on morphological characteristics.

Emergence of uncoated sugar beet seeds planted in the Pythium-infested soil used in the indoor experiment was reduced by 37% (21% emergence) compared with seeds planted in non-infested soil (58% emergence). Pythium was reisolated from 65% of the non-germinated seeds tested. Despite the lack of clear antagonism against Pythium sp. “group G” in the in vitro assays, seed treatment with the Rhizobium strains significantly (P<0.05) increased emergence of sugar beet in soil artificially infested with Pythium sp. “group G” compared with the untreated control (Table 2). The most effective strains for biological control of damping-off of sugar beet were R3, R4, R5, R7, R12, R20, and R21 (Table 2).

TABLE 2
Control of Pythium damping-off of sugar beet (Beta vulgaris) by seed
treatment with R. leguminosarum bv. viceae (indoor experiments).
Rhizobium leguminosarum bv. viceae

| Strain | Emergence (%) |
|---|---|
| R12 | 52a |
| R20 | 46ab |
| R21 | 44ab |
| R4 | 43abc |
| R3 | 42abc |
| R7 | 42abc |
| R5 | 41abc |
| R9 | 39bcd |
| R8 | 36bcd |
| R19 | 32cd |
| Untreated control | 21e |

Note:
Emergence of sugar beet seedlings was determined 14 d after planting. Means are of three replicates from three independent experiments. All experiments gave similar results. Means followed by the same letter are not significantly different at P = 0.05 level (Fisher's LSD test).

### Example 6

### Control of Pythium Damping-Off of Sugar Beet and Field Pea by Rhizobium Leguminosarum bv. Viceae Strains (Field Experiments)

The selected strains of R. leguminosarum bv. viceae (R12, R20, and R21) effective against Pythium damping-off of sugar beet in indoor experiments were tested for control of damping-off of sugar beet and field pea in fields naturally infested with Pythium spp. at the Lethbridge Research Centre, Alberta. The efficacy of the Rhizobium strains was compared with the biocontrol agent P. fluorescens 708, which was shown to improve emergence of sugar beet, field pea, canola, and safflower in soil naturally infested with Pythium spp. (Bardin et al. 2003). The seeds were coated with the bacterial slurry as described previously using 2.4 and 9.5 mL bacterial slurry/100 seeds of sugar beet and field pea, respectively. The seeds were dried overnight at room temperature on a metallic mesh, which was placed on a paper towel to absorb the excess slurry. The number of bacteria coated onto the seeds was similar for the four bacterial strains, ranging from 1.4×10⁶±0.2×10⁶ to 2.3×10⁷±0.2×10⁷ cfu/seed for sugar beets and 3.0×10⁷±0.3×10⁷ to 1.2×10⁸±0.4×10⁸ cfu/seed for field peas. The coated seeds were then stored at 4° C. until planting. Bacterial counts on the seeds were determined by vortexing five coated seeds in 5 mL of distilled sterile water for 30 seconds, and by plating serial dilutions on TY agar medium in Petri dishes for Rhizobium strains and on PDA in Petri dishes for P. fluorescens. Each bacterial count was performed in duplicate, and bacterial determinations for each treatment were performed twice. The Rhizobium-treated and untreated seeds were machine seeded into 0.9 m wide×5.0 m long plots made of 4 rows of 100 seeds/row in a field naturally infested with Pythium spp. The plots were trimmed to 3.5 m after all seedlings emerged. Treatments were arranged in a randomized complete block design, with six replicates per treatment. The field experiments were performed twice, once in May and again in August 2001 in Fairfield Farm, Lethbridge, Alberta. Seedling emergence was recorded 4 weeks after planting and was compared with the uninoculated and fungicide controls. The amount of Thiram™ for the fungicide-treated seeds was 90 g/25 kg sugar beet seeds and 30 g/25 kg field pea seeds.

Treatment of pea seeds with R. leguminosarum bv. viceae strain R12 or R20 caused a significant (P<0.05) increase in seedling emergence compared with the untreated control in the two field experiments (Table 3). The efficacy of the two Rhizobium strains was similar to that of seed treatments with the rhizobacterium P. fluorescens 708. Rhizobium leguminosarum bv. viceae R21 significantly increased pea seedling emergence compared with the untreated control in the second (August 2001) but not in the first (May 2001) field experiment. The level of seedling emergence in the second field experiment was lower but not significantly (P>0.05) different from that of R. leguminosarum bv. viceae R20 and P. fluorescens 708. None of the bacterial treatments were as effective as the fungicide Thiram™ for control of damping-off of field peas.

In the sugar beet experiments conducted in May and August of 2001, seed treatment with R. leguminosarum bv. viceae R12, R20, or R21 increased seedling emergence compared with the untreated control (Table 3). This increase was significant (P<0.05) in the August experiment. In both the May and August field experiments, the percent emergence of the Rhizobium-treated seeds was not significantly different from that of seeds treated with P. fluorescens 708. Rhizobium leguminosarum bv. viceae R12 and R21 were as effective as the fungicide treatment for protection of sugar beet seedlings against Pythium damping-off in the August field experiment, while P. fluorescens 708 was as effective as the fungicide treatment in the May field experiment (Table 3).

TABLE 3
Effect of bacterial seed treatment on field pea and sugar beet
emergence in a field naturally infested with Pythium spp.

| | Emergence (%) | | | |
|---|---|---|---|---|
| | Pea | | Sugar beet | |
| | (Pisum sativum) | | (Beta vulgaris) | |
| | May | August | May | August |
| Treatment | 2001 | 2001 | 2001 | 2001 |
| Untreated control | 41.4d | 12.8d | 10.0c | 6.5d |
| Fungicide (Thiram ™)* | 71.4a | 48.3a | 28.6a | 27.0a |
| R12 | 54.3bc | 34.4b | 18.6bc | 24.7abc |
| R20 | 51.4c | 30.6bc | 14.3bc | 21.9c |
| R21 | 37.1d | 23.7c | 17.1bc | 24.9ab |
| Pseudomonas fluorescens 708 | 60.0b | 29.6bc | 20.0ab | 23.5bc |

Note:
Seedling emergence was determined 4 weeks after planting. Values are means of six replicates. Means within each column followed by the same letter are not significantly different at P = 0.05 level (Fisher's LSD test).
*The concentration of Thiram ™ was 90 g/25 kg sugar beet seeds and 30 g/25 kg field peas.

### Example 7

### Control of Pythium Damping-Off of Pea and Lentil by Rhizobium Strains (Field Experiments)

The strains of Rhizobium leguminosarum bv. viceae used for the study were 99A1, R12, R20, and R21. Strain 99A1 was originated from the commercial pea inoculant produced by Agrium, Inc. Calgary, Alberta. Bacterial cultures were grown on tryptone-yeast agar (TYA) (Beringer, 1974) in Petri dishes for 48 h at room temperature (20±2° C.). The resulting colonies were suspended in 5 ml per dish of 1% methyl cellulose (Sigma-Aldrich, Milwaukee, Wis.) in sterile distilled water, and scraped gently with a spatula to obtain bacterial slurries. Seeds of field pea cv. Trapper and lentil cv. Laird were soaked for 20 minutes in the slurries, spread on a metallic mesh sheet with paper towel underneath to absorb the excess slurry, and air-dried overnight under a fume hood. Enumeration of bacteria coated onto seeds was done by placing 5 seeds in a test tube with 5 ml of sterile distilled water, vortexing for 30 sec, and plating serial dilutions on TYA, 0.1 ml per 9-cm dish. After incubation at room temperature for 3 days, bacterial colonies developed in each dish were counted. There were two replicates for each treatment.

Field experiments were conducted at the Agriculture and Agri-Food Canada Research Centre near Lethbridge, Alberta, Canada, in a field naturally infested with Pythium spp. (predominantly Pythium sp. ‘group G’). For the pea experiment, seeds were planted using a plot seeder on 28 May 2004, in 4-row plots with a row length of 5 m, a row spacing of 22.5 cm, and a plant spacing of 5 cm (i.e. 20 seeds/m). Untreated seeds and fungicide-treated seeds (Thiram™ at the rate of 30 g/25 kg seed) (Gustafson; Calgary, Alberta, Canada) were used as controls. Treatments were arranged in a randomized block design with 6 replicates. For the lentil experiment, seeds were planted on the same date and using the same parameters as for field pea.

Seedling emergence for each plot was determined. The number of healthy seedlings and the number of wilted seedlings were counted in the middle 3 m of each row, and the percent loss due to pre-emergent and post-emergent damping-off were calculated, as well as the final stand establishment. The causal agent of seedling death was determined by collecting 10 non-emerged seedlings and all of the wilted seedlings from each plot, washing in running water, surface sterilizing in 70% ethanol for 90 sec, incubating on potato dextrose agar (PDA) in Petri dishes at room temperature for 7 days, and examining the organisms derived from each sample. Results of reisolation of diseased seedlings were used to calculate the incidence of damping-off due to Pythium spp. for each plot.

Seedling height for each plot was determined (6-node stage for peas; 5-node stage for lentils). For each row, ten seedlings were randomly selected and the distance from the first node to the terminal branch of each seedling was measured.

Differences between treatments for incidence of damping-off, seedling emergence and seedling height data were analyzed for statistical significance using analysis of variance (ANOVA) and means of treatments for each set of data were separated using Duncan's multiple range test at the P=0.05 level. All statistical analyses were done using SAS Statistical Analysis Software, Version 8.2 (SAS Institute Inc., Cary, N.C. 2001). The results are set forth in Tables 4-7.

TABLE 4
Effect of seed treatment with Rhizobium leguminosarum biovar
viceae strains on damping-off of field pea.

| | Damping-off (%)¹ | | | Final |
|---|---|---|---|---|
| Treatment | Pre-emergent | Post-emergent | by Pythium | Stand (%)¹ |
| Control | 62.0 a² | 0.2 | 55.4 a² | 37.8 a² |
| 99A1 | 62.0 a | 0.3 | 54.8 a | 37.7 a |
| R12 | 55.2 ab | 0.1 | 47.0 ab | 44.7 ab |
| R20 | 51.6 b | 0.7 | 43.4 b | 47.9 b |
| R21 | 28.5 c | 0.8 | 23.7 c | 70.7 c |
| Fungicide | 20.3 c | 0.6 | 16.7 c | 79.1 c |
| (Thiram) | | | | |

¹Based on 60 seeds planted per 3-meter section of row, 4 rows per plot.
²Means within each column followed by the same letter are not significantly different (Duncan's multiple range test; P > 0.05).

TABLE 5
Effect of seed treatment with Rhizobium leguminosarum biovar
viceae strains on seedling height of field pea.

| Treatment | Plant Height (cm)¹ |
|---|---|
| Control | 11.1 a² |
| R20 | 11.1 a |
| 99A1 | 12.2 ab |
| R12 | 12.8 b |
| R21 | 12.8 b |
| Fungicide (Thiram) | 14.6 c |

¹Distance from the first node to the terminal branch; measured at the 6-node stage (4 weeks after planting). Based on random selection of 10 seedlings per row, 4 rows per plot.
²Means within each column followed by the same letter are not significantly different (Duncan's multiple range test; P > 0.05).

TABLE 6
Effect of seed treatment with Rhizobium leguminosarum biovar viceae
strains on damping-off of lentil.

| | Damping-off (%)¹ | | | |
|---|---|---|---|---|
| | Pre- | Post- | | Final |
| Treatment | emergent | emergent | by Pythium | Stand (%)¹ |
| 99A1 | 50.6 a² | 2.3 | 38.1 a² | 47.1 a² |
| Control | 46.3 ab | 2.7 | 35.3 ab | 51.0 ab |
| R21 | 44.1 ab | 1.5 | 31.5 bc | 54.4 bc |
| R20 | 43.1 b | 3.3 | 30.2 bc | 53.6 bc |
| R12 | 39.9 bc | 2.2 | 27.8 cd | 57.9 cd |
| Fungicide (Thiram) | 34.2 c | 2.6 | 22.8 d | 63.2 d |

¹Based on 60 seeds planted per 3-meter section of row, 4 rows per plot.
²Means within each column followed by the same letter are not significantly different (Duncan's multiple range test; P > 0.05).

TABLE 7
Effect of seed treatment with Rhizobium leguminosarum biovar
viceae strains on seedling height of lentil.

| Treatment | Plant Height (cm)¹ |
|---|---|
| Control | 11.2 a² |
| R20 | 11.2 a |
| 99A1 | 11.2 a |
| R12 | 11.3 a |
| R21 | 11.3 a |
| Fungicide (Thiram) | 11.4 a |

¹Distance from the first node to the terminal branch; measured at the 5-node stage (4 weeks after planting). Based on random selection of 10 seedlings per row, 4 rows per plot.
²Means within each column followed by the same letter are not significantly different (Duncan's multiple range test; P > 0.05).

Enumeration of bacteria coated onto seeds revealed similar numbers of bacteria per seed for all four strains of R. leguminosarum bv. viceae, for both field pea and lentil. The number of colony-forming units (cfu) per seed ranged from 2.3×10⁵ to 2.9×10⁵ for pea, and from 2.2×10⁵ to 5.1×10⁵ for lentil.

Reisolation of diseased pea seedlings revealed that 84% of the seedlings killed by pre- and post-emergent damping-off were infected with Pythium spp., whereas the remaining seedlings were colonized by Fusarium spp. Treatment of pea seeds with R20, R21 or Thiram™ significantly (P<0.05) reduced pre-emergent damping-off compared to the untreated control (Table 4). The incidences of pre-emergent damping-off for the treatments of R20, R21 and Thiram™ were 51.6%, 28.5% and 20.3%, respectively, compared to 62.0% for the untreated control. There was no significant difference in incidence of pre-emergent damping-off between the treatments of R21 and Thiram™. Damping-off losses of pea due to Pythium spp. alone followed a similar trend, ranging from 16.7% in the fungicide treatment and 23.7% in the treatment of R21, to 55.4% in the untreated control (Table 4). The height of pea plants arising from seed treated with R12, R21 or Thiram™ was significantly (P<0.05) greater than for plants arising from untreated seed (Table 5). Seedling height for the treatments of R12 and R21 was 12.8 cm for both Rhizobium strains, compared to 14.6 cm for the treatment of Thiram™, and 11.1 cm for the untreated control.

For the lentil experiment, results of plating of diseased seedlings showed that 68% were infected with Pythium spp., 22% were infected with Botrytis cinerea, and the remainder was colonized by Fusarium spp. Treatment of lentil seeds with R20, R12 or Thiram™ significantly (P<0.05) reduced incidence of pre-emergent damping-off compared to the untreated control (Table 6). The disease incidences for the treatments of R20, R12 and Thiram™ were 43.1%, 39.9% and 34.2%, respectively, compared to 50.6% for the untreated control. Incidence of damping-off of lentil due to Pythium spp. alone followed the same trend, ranging from 22.8% in the fungicide treatment and 27.8% in the treatment of R12, to 38.1% in the untreated control (Table 6). No significant differences in seedling height of lentil were detected among the treatments (Table 7).

Among the four strains of R. leguminosarum bv. viceae tested, strains R20 and R21 from pea were most effective for control of damping-off of pea (Table 4), whereas the strain R12 from lentil was most effective for control of damping-off of lentil (Table 6). The study on pea also suggests that the strains may have a growth promoting effect on seedlings, as seen in the case of increased height of pea seedlings for the treatments of R12 and R21 (Table 5).

All references discussed herein are incorporated by reference. One skilled in the art will readily appreciate that the present invention is well adapted to carry out the objects and obtain the ends and advantages mentioned, as well as those inherent therein. The present invention may be embodied in other specific forms without departing from the spirit or essential attributes thereof and, accordingly, reference should be made to the appended claims, rather than to the foregoing specification, as indicating the scope of the invention.

### REFERENCES

Bardin, S. D., and Huang, H. C. 2001. Survey of damping-off diseases of sugar beet in southern Alberta in 2000. Can. Plant Dis. Surv. 1:136-137.
Bardin, S. D., Huang, H. C., Liu, L., and Yanke, L. J. 2003. Control, by microbial seed treatment, of damping-off caused by Pythium sp. on canola, safflower, dry pea and sugar beet. Can. J. Plant Pathol. 25:268-275.
Beringer, J. E. 1974. R-factor transfer in Rhizobium leguminosarum. J. Gen. Microbiol. 84:188-198.
Brockwell, J., Bottomly, P. J., and Thies, J. E. 1995. Manipulation of rhizobia microflora for improving legumes productivity and soil fertility: a critical assessment. Plant Soil, 174:143-180.
Dileep Kumar, B. S., Berggren, I., and Martensson, A. M. 2001. Potential for improving pea production by co-inoculation with fluorescent Pseudomonas and Rhizobium. Plant Soil, 229:25-34.
Drapeau, R., Fortin, J. A., and Gagnon, C. 1973. Antifungal activity of Rhizobium. Can. J. Bot. 51:681-682.
Dunne, C., Crowley, J. J., Moenne-Loccoz, Y., Dowling, D. N., de Bruijn, F. J., and O'Gara, F. 1997. Biological control of Pythium ultimum by Stenotrophomonas maltophilia W81 is mediated by an extracellular proteolytic activity. Microbiology, 143:3921-3931.
Estevez de Jensen, C., Percich, J. A., and Graham, P. H. 2002. The effect of Bacillus subtilis and Rhizobium inoculation of dry bean seed on root rot severity and yield in Minnesota. Annu. Rep. Bean Improv. Coop. 45:98-99.
Huang, H. C., Morrison, R. J., Mündel, H.-H., Barr, D. J. S., Klassen, G. R., and Buchko, J. 1992. Pythium sp. “group G”, a form of Pythium ultimum causing damping-off of safflower. Can. J. Plant Pathol. 14:229-232.
Hwang, S. F., and Chang, K. F. 1989. Root rot disease complex of field pea in north-eastern Alberta in 1988. Can. Plant Dis. Surv. 69:139-141.
Leonard, L. T. 1943. A simple assembly for use in testing cultures of rhizobia. J. Bacteriol. 45:523-527.
Martin, F. N., and Loper, J. E. 1999. Soilborne plant diseases caused by Pythium spp.: ecology, epidemiology, and prospects for biological control. Crit. Rev. Plant Sci. 18:111-181.
Miller, J. H. 1972. Experiments in molecular genetics. Cold Spring Harbor Laboratory, Cold Spring Harbor, N.Y.
Nautiyal, C. S. 1997. Rhizosphere competence of Pseudomonas sp. NBRI9926 and Rhizobium sp. NBRI9513 involved in the suppression of chickpea (Cicer arietinum L.) pathogenic fungi. FEMS Microbiol. Ecol. 23:145-158.
Omar, S. A., and Abd-Alla, M. H. 1998. Biocontrol of fungal root rot diseases of crop plants by the use of rhizobia and bradyrhizobia. Folia Microbiol. 43:431-437.
Ozkoc, I., and Deliveli, M. H. 2001. In vitro inhibition of the mycelial growth of some root rot fungi by Rhizobium leguminosarum biovar phaseoli isolates. Turk J. Biol. 25:435-445.
Siddiqui, I. A., Ehteshamul-Haque, S., Zaki, M. J., and Ghaffar, A. 2000. Greenhouse evaluation of rhizobia as biocontrol agent of root-infecting fungi in okra. Acta Agrobot. 53:13-22.
Simpfendorfer, S., Harden, T. J., and Murray, G. M. 1999. The in vitro inhibition of Phytophthora clandestina by some rhizobia and the possible role of Rhizobium trifolii in biological control of Phytophthora root rot of subterranean clover. Aust. J. Agric. Res. 50:1469-1473.

Vincent, J. M. 1970. A manual for the practical study of root nodule bacteria (IBM Handbook No. 15). Blackwell Scientific Publications, Oxford, UK.

We claim:

1. A method for treating or protecting a plant susceptible to Pythium ultimum comprising contacting the plant or part thereof, or the soil surrounding the plant, with an effective amount of at least one Rhizobium leguminosarum biovar viceae strain which has suppressive activity against Pythium ultimum.

2. The method according to claim 1 wherein the at least one Rhizobium leguminosarum biovar viceae strain which has suppressive activity against Pythium sp. “group G”.

3. The method according to claim 1 wherein the plant is in the form of a seed.

4. The method according to claim 1 wherein the plant part contacted comprises root.

5. The method according to claim 1 wherein the plant is selected from the group consisting of sugar beet, field pea, lentil, safflower, canola, chickpea, sunflower, alfalfa, soybean and field bean.

6. The method according to claim 1 wherein the plant is treated or protected from seed rot or damping-off.

7. The composition according to claim 2 wherein the strain inhibits the colonization of Pythium sp. “group G” on an agar plate.

8. The method according to claim 1 wherein the bacteria comprises the bacterium deposited under International Depository Authority of Canada Accession number IDAC 200704-01.

9. The method according to claim 1 wherein the bacteria comprises the bacterium deposited under International Depository Authority of Canada Accession number IDAC 200704-02.

10. The method according to claim 1 wherein the bacteria comprises the bacterium deposited under International Depository Authority of Canada Accession number IDAC 200704-03.

11. The method according to claim 1 wherein the bacteria comprises the bacterium deposited under International Depository Authority of Canada Accession number IDAC 200704-04.

12. The isolated Rhizobium leguminosarum biovar viceae strain deposited under International Depository Authority of Canada Accession number IDAC 200704-01.

13. The isolated Rhizobium leguminosarum biovar viceae strain deposited under International Depository Authority of Canada Accession number IDAC 200704-02.

14. The isolated Rhizobium leguminosarum biovar viceae strain deposited under International Depository Authority of Canada Accession number IDAC 200704-03.

15. The isolated Rhizobium leguminosarum biovar viceae strain deposited under International Depository Authority of Canada Accession number IDAC 200704-04.

16. An antifungal composition comprising bacteria of at least one isolated Rhizobium leguminosarum biovar viceae strain which has suppressive activity against Pythium ultimum.

17. The composition according to claim 16 wherein the at least one Rhizobium leguminosarum biovar viceae strain has suppressive activity against Pythium sp. “group G”.

18. The composition according to claim 17 wherein the strain inhibits the colonization of Pythium sp. “group G” on an agar plate.

19. The composition according to claim 16 comprising one or more biologically inert components.

20. The composition according to claim 19 wherein the one or more inert component is selected from the group consisting of carrier materials, stickers, binders, adhesives, extenders, and mixtures thereof.

21. The composition according to claim 19 comprising cells or spores of other biological control agents, one or more chemical fungicides, or one or more pesticides.

22. The composition according to claim 16 wherein the bacteria strain is the Rhizobium leguminosarum biovar viceae strain deposited under International Depository Authority of Canada Accession number IDAC 200704-01.

23. The composition according to claim 16 wherein the bacteria strain is the Rhizobium leguminosarum biovar viceae strain deposited under International Depository Authority of Canada Accession number IDAC 200704-02.

24. The composition according to claim 16 wherein the bacteria strain is the Rhizobium leguminosarum biovar viceae strain deposited under International Depository Authority of Canada Accession number IDAC 200704-03.

25. The composition according to claim 16 wherein the bacteria strain is the Rhizobium leguminosarum biovar viceae strain deposited under International Depository Authority of Canada Accession number IDAC 200704-04.
